(19) United States
(12) Patent Application Publication
(10) Pub. No.: US 2022/0080068 A1
(43) Pub. Date: Mar. 17, 2022

(54) BIO-DECONTAMINATION SYSTEM AND METHOD USING OZONE

(71) Applicant: Ground Effects Environmental Services Inc., Regina (CA)

(72) Inventors: Sean FRISKY, Regina (CA); Jason BEAUDIN, Regina (CA)

(21) Appl. No.: 17/314,441

(22) Filed: May 7, 2021

Related U.S. Application Data
(60) Provisional application No. 63/076,992, filed on Sep. 11, 2020.

(51) Int. Cl. A61L 2/20; C01B 13/11; B01D 53/00; B01D 53/66; A61L 9/12; F24F 3/16

(52) U.S. Cl. CPC A61L 2/202; F24F 2221/125; B01D 53/007; B01D 53/66; A61L 9/122; F24F 3/16; B01D 2257/106; B01D 2259/10; B01D 2259/804; A61L 2202/11; A61L 2209/212; B01D 2257/91; B01D 2251/104; B01D 2259/4508; C01B 13/11

(57) ABSTRACT

An ozone generating system, a controller and a method for generating ozone in an enclosed space is provided. The system can include a housing, an intake vent, an exhaust vent, a blower provided in the housing, an ozone generator to route generated ozone into the enclosed space and an ozone sensor. The controller and method can include adding ozone to air in an enclosed space to form ozone enriched air, until a set ozone target level is reached and maintaining the ozone level in the enclosed space in a desired ozone range for a treatment time period.

The present invention relates to a device and method for virus reduction and infectivity and/or pest control and more particularly to devices and methods that utilize ozone to treat an enclosed space.

### BACKGROUND

SARS-COVID-2 and other viruses can spread relatively fast across a large region and can cause a regional epidemic and in more severe cases, a global pandemic. When a person has become infected with such a virus, it is desirable or even required to thoroughly sanitize any indoor spaces they have been where other people may have access before other people are allowed to go back to the space. These could be office buildings, warehouses and production facilities, public transit, universities, libraries, gymnasiums, sporting arenas, etc.

For example, if an employee who works in a small office building tests positive for such a virus, along with the employee being quarantined, the small office building should be thoroughly sanitized/disinfected before other people re-enter the facility to prevent the virus being passed, by dermal contact or by way of aerosol transmission, to others who may enter the small office building and causing other employees to contract the virus.

However, this sanitation/disinfection requires each surface and airborne particle in a space be treated to kill any pathogens that may be present. If this is being done by manually wiping down each surface with disinfectant, not only is it ineffective in destroying the airborne aerosols, yet also the time involved to manually wipe down each surface can be considerable and delay people from being able to return back to the space in a reasonable time frame. Additionally, manually wiping down every surface in a space with disinfectant often involves using harsh chemicals that are not only undesirable to handle, but can leave unwanted residue for people re-entering the space.

Using ozone is one way of disinfecting these spaces without using chemicals. However, the use of ozone comes with its own challenges. Ozone has the potential to be dangerous to people and other living things at higher concentrations so it must be dealt with in a safe manner. Therefore, it is desirable to have an ozone generating system and method that can be placed in an environment such that it can easily and safely treat the space without risking harm to people, yet sufficiently disinfect and deactivate viruses in that space.

### SUMMARY OF THE INVENTION

In an aspect, an ozone generating system for generating ozone in an enclosed space is provided. The system comprising: a housing having a front end, a back end, a first side, a second side, a top, a bottom, an intake vent passing through the housing, an exhaust vent passing through the housing, a blower provided in the housing, an intake pipe provided in the housing and routing air entering the housing through the intake vent to the blower, an exhaust pipe provided in the housing and operably connected between the blower and the exhaust vent, an ozone generator provided in the housing and operably connected to the exhaust pipe to route ozone generated by the ozone generator into the exhaust pipe, and an ozone sensor operably connected to the controller and operative to measure an amount of ozone in the air surrounding the ozone sensor.

In a further aspect, the system can have a controller having: at least one processing unit; an input interface operatively connected to the ozone sensor; an output interface operatively connected to the blower and the ozone generator; and at least one memory containing program instructions. The at least one processing unit can be, responsive to the program instructions, operative to: initiate a start delay time; send a signal to the blower to turn on the blower and circulate air from the enclosed space surrounding the system through the system before exhausting the air to the enclosed space; when the start delay time has passed, send a signal to the ozone generator to turn on the ozone generator and routing ozone to the air circulating through the system to form ozone enriched air; continuing to enrich air circulating through the system with ozone and exhausting the ozone enriched air to the enclosed space, until a signal is received from the ozone sensor indicating an ozone level in the air in the enclosed space has reached a set ozone target level; maintaining an ozone level in the air in the enclosed space in a desired ozone range for a treatment time period; and after the treatment time period has passed, sending a signal to the ozone generator to turn off the ozone generator.

In another aspect, a method of generating ozone in an enclosed space is provided. The method comprising: after a start delay time period, adding ozone to air in an enclosed space to form ozone enriched air; continuing to enrich the air in an enclosed space with ozone, until the ozone in the air in the enclosed space reaches a set ozone target level; after the ozone in the air in the enclosed space reaches the set ozone target level, maintaining an ozone level in the air in the enclosed space in a desired ozone range for a treatment time period; and after the treatment time period has passed, stopping adding any ozone to the air in the enclosed space.

### DESCRIPTION OF THE DRAWINGS

A preferred embodiment of the present invention is described below with reference to the accompanying drawings, in which:

FIG. 1 is a front perspective view of an ozone generating system;

FIG. 2 is a rear perspective view of the ozone generating system of FIG. 1;

FIG. 3 is a first side perspective view of the ozone generating system of FIG. 1;

FIG. 4 is second side perspective view of the ozone generating system of FIG. 1;

FIG. 5A is a front view of an electrolytic oxidation device that could be used in the ozone generating system;

FIG. 5B is a side view the electrolytic oxidation device shown in FIG. 6a;

FIG. 6A is a perspective view of a section of exhaust pipe with a container for containing an electrolytic oxidation device;

FIG. 6B is an exploded sectional view of the container shown in FIG. 6A;

FIG. 7 is a schematic illustration of a control system that can control the operation of an ozone generating system; and

FIGS. 8-10 are a flowchart of a method for disinfecting the enclosed space surrounding the system.

### DETAILED DESCRIPTION OF THE ILLUSTRATED EMBODIMENTS

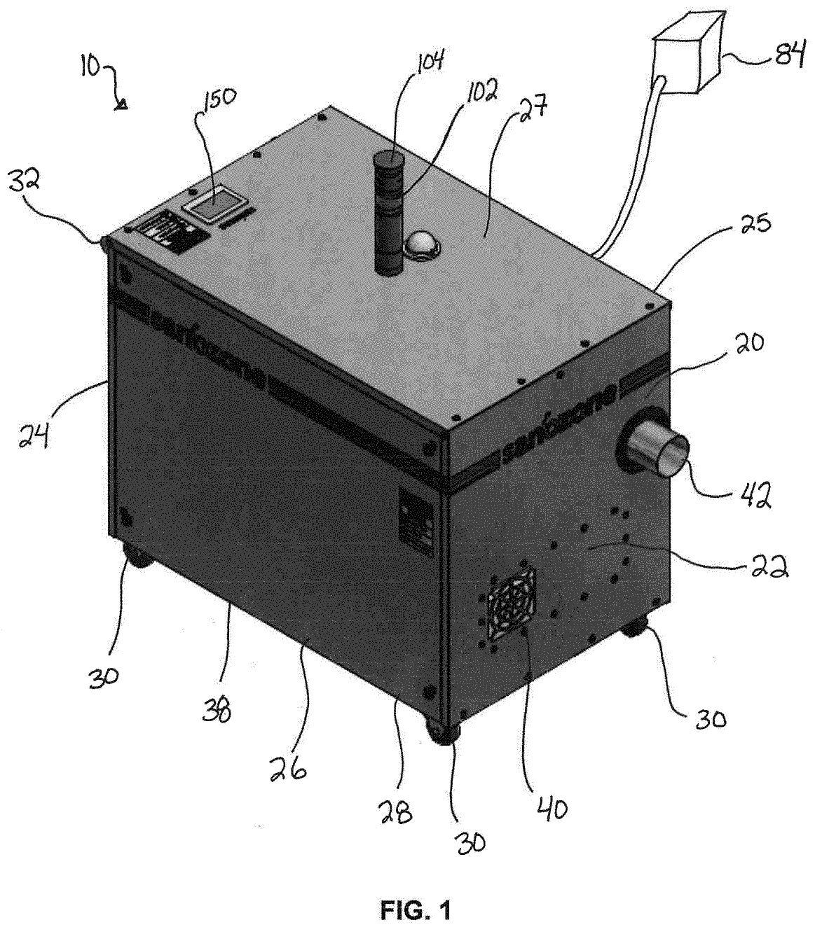

FIGS. 1-4 illustrate an ozone generating system 10 for reducing the infectivity, and even eliminating, SARS-COVID-2, as well as other airborne viruses, from surfaces in unoccupied enclosed spaces, such as office spaces, hotels, laboratories, casinos, arena, etc. In a further aspect, the ozone generating system 10, could be used for pest control to reduce or even eliminate pest in an enclosed space, such as cockroaches, bed bugs, mice and other vermin, etc. The system 10 generates ozone and mixes the ozone gas with ambient air from the enclosed space to achieve an elevated level of ozone in the air, to treat viruses and other contaminants. The system 10 generates ozone and mixes the ozone gas with ambient air from the enclosed space until a pre-determined ozone level is measured in the enclosed space. Once the pre-determined ozone level is met, the system 10 keeps the ozone concentration in the room in a desired ozone range for a pre-determined treatment time period, after which pre-determined dwell time has passed the system 10 proceeds into destruction mode where ozone levels are quickly brought down in the enclosed space to suitable levels for occupancy.

Referring to FIGS. 1-4, the system 10 can include: a housing 20; an intake vent 40; an exhaust vent 42; a UV light chamber 50 with a UV light 52; a blower 60; an intake pipe 70; an exhaust pipe 80; an ozone generator 90; an air filter 94; an oxygen generator 82; an ozone sensor 84; a controller 300; and, a control panel 150.

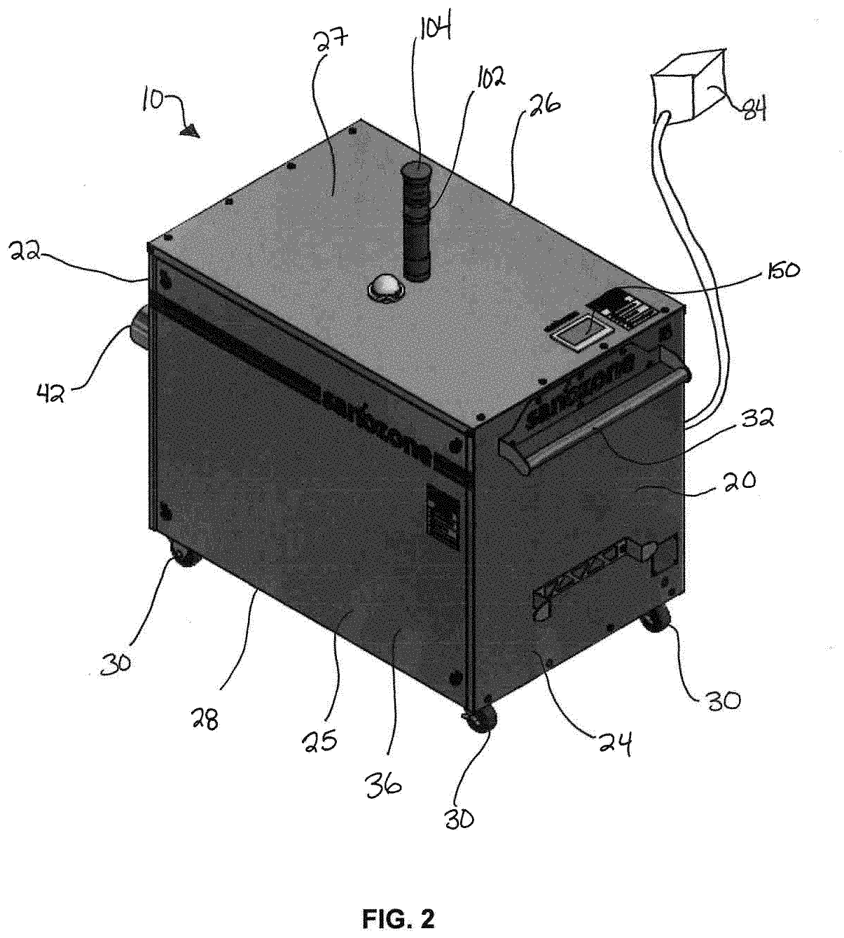

Referring to FIGS. 1 and 2, the housing 20 can contain or house all the internal components and have a front end 22, a back end 24, a first side 25, a second side 26, a top 27 and a bottom 28, such as a cabinet. Swivel casters 30 can be provided attached to the bottom 28 of the housing 20 to allow the system 10 to be wheeled around by a user and a handle 32 can be provided on the rear end 24 proximate a top of the rear end 24 to allow the system 10 to be pushed by a user.

The housing 20 can have a removable first access panel 36 on the first side 25 and a removable second access panel 38 on the second side 26 to allow a person to access the internal components.

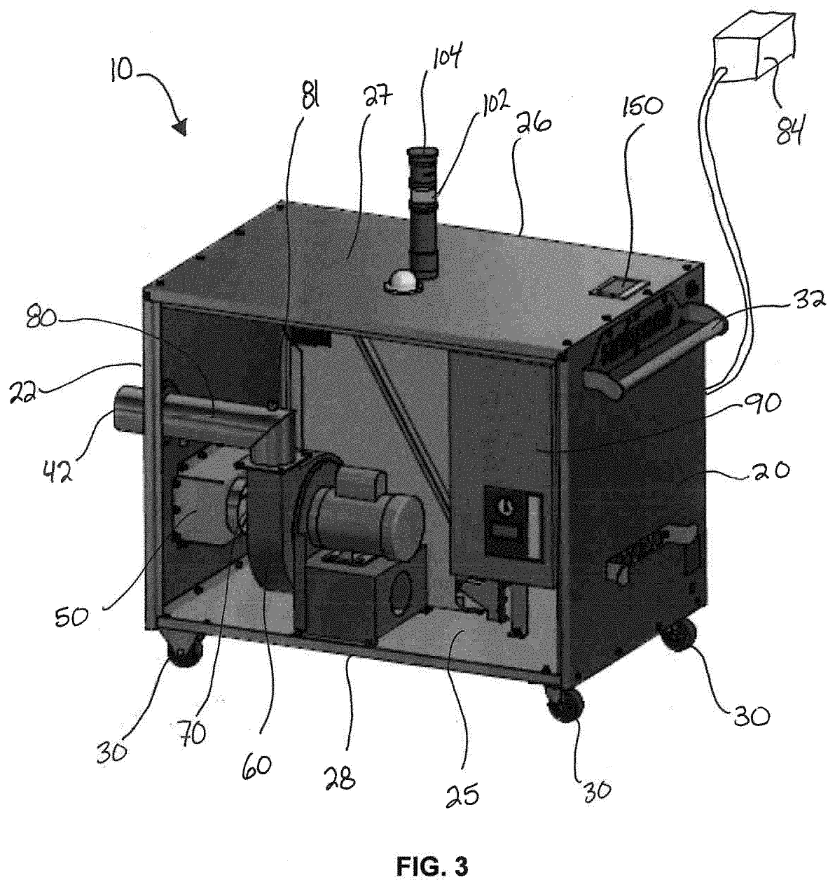

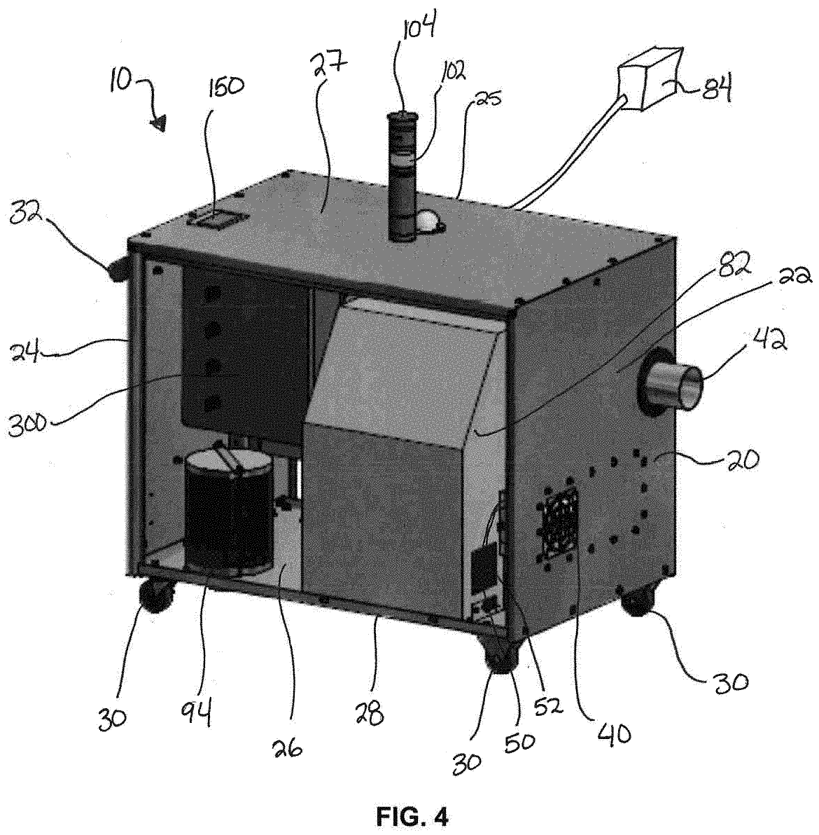

Referring to FIGS. 3 and 4, the housing 20 can house internal components such as the UV light chamber 50, the blower 60, the ozone generator 90, the air filter 94, the oxygen generator 82, the controller 300 and the control panel 150.

The intake vent 40 can be positioned on the front end 22 of the housing 20 and allows air from the enclosed space surrounding the system 10 to enter the housing 20 and pass into the UV light chamber 50.

The UV light chamber 50 contains the UV Light 52 to illuminate the UV light chamber 50 with UV-C light. The UV light 52 can be switched on and off and is used to treat the incoming air that is passing through the UV light chamber 50 during destruction mode to break down or “destroy” ozone in this air. The UV light 52 causes disassociation of the ozone (O³) by breaking one of the oxygen bonds in the ozone molecules. This causes the ozone (O³) to break down into oxygen (O²). When the UV light 52 is turned on and shining in the UV light chamber 50, the UV light comes into contact with the air that is passing through the UV light chamber 50 and can break down ozone in this air into oxygen. When the UV light 52 is turned off, the air will pass through the UV light chamber 50 without any of the ozone in the air being broken down into oxygen by the UV light 52. Alternatively, the design could include, or alternatively use, a catalyst for accelerating ozone destruction.

From the UV light chamber 50, the air is routed through the intake pipe 70 to the blower 60.

The blower 60 can be a centrifugal blower with an impeller and a blower housing to suck air into the blower 60 and discharge it to the exhaust pipe 80 leading to the exhaust vent 42. High concentrated ozone can be added from the ozone generator 90 into the exhaust pipe 80 at the inlet port 81 for rapid dispersion and mixing of the concentrated air passing through the exhaust pipe 80 prior to air exiting the system 10 through the exhaust vent 42. When the blower 60 is operating, air surrounding the system 10 can be sucked into the intake vent 40 through the UV chamber 50 and into the blower 60 before the air is discharged through the exhaust pipe 80 and out the exhaust vent 42 to be discharged back into the air surrounding the system 10.

The ozone generator 90 can be provided in the housing 20 and capable of generating ozone by converting oxygen from the oxygen generator 82 to ozone. The ozone generator 90 can route ozone generated by the ozone generator 90 downstream from the blower 60 to inlet port 81 and be introduced into the air being discharged from the system 10 through the exhaust vent 42.

The ozone sensor 84 can be connected to the controller 300, either through a wired connection or a wireless connection, so that the ozone sensor 84 can be placed in the enclosed space some distance from the housing 20 and the other components and measure the amount of ozone in the air surrounding the ozone sensor 84. In this manner, the controller 300 can monitor the ozone in the air in the enclosed space at a distance from the housing 20 (where the ozone sensor 84 is positioned) to get a more accurate measurement of the ozone in the air in the enclosed space. In one aspect, the ozone sensor 84 can be wired to the controller 300 by a 50 meter length of cable.

A beacon 102 can be provided on the top 27 of the housing 20 or remotely mounted in a location that provides greater visibility. The beacon 102 can be a light to visually warn anyone near the system 10 that the system 10 is operating by lighting the beacon 102.

A sounder 104 can be provided in the housing 20 to audibly warn anyone near the system 10 that the system is operating by generating an alarm or other audible frequency.

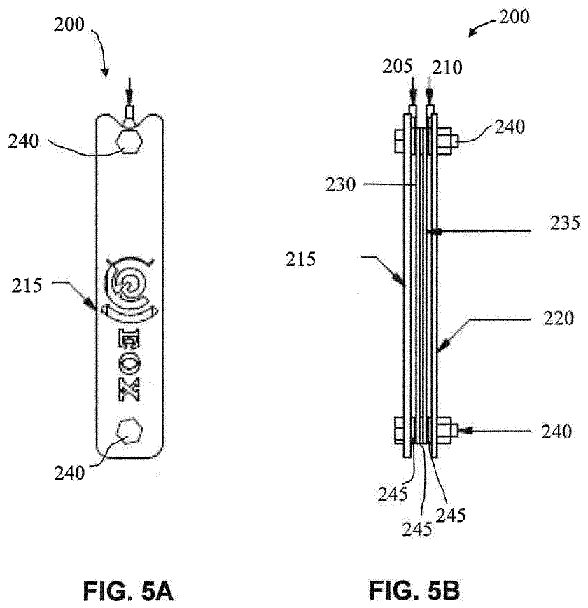

Referring to FIGS. 5A and 5B, in one aspect, an electrolytic oxidation device 200 can be provided in the system 10 that generates ozone and hydroxyl free radicals and these ozone and hydroxyl free radicals, along with vaporized liquid, can then pass into the exhaust pipe 80 to mix with the air passing through the exhaust pipe 80. This adds more ozone and hydroxl free radicals to the air in the enclosed space, but the vaporized liquid from the electrolytic oxidation device 200 can also increase the humidity in the air in the enclosed space helping with the disinfection/virus deactivation of the enclosed space by the ozone generated by the system 10.

The electrolytic oxidation device 200 can include: a positive terminal 205; a negative terminal 210; a front cover 215; a back cover 220; an anode 230; a cathode 235; fasteners 240; and spacers 245.

The positive terminal 205 can be connected to the anode 230 which can be a metal plate, such as a titanium plate. The negative terminal 210 can be connected to the cathode 235 which can also be a metal plate, such as a titanium plate, stainless steel or alternative material.

The front cover 215 and the back cover 220 can be formed of a non-electrically conductive material such as ultra-high-molecular-weight polyethylene (UHMW).

The fasteners 240 can be used to secure the front cover 215, anode 230, cathode 235 and back cover 220 in series with the spacers 245 positioned on the fasteners 245 to space the anode 230 and cathode 235, as well as space the front cover 215 and back cover 220 from the anode 230 and cathode 235. In one aspect the, fasteners can be bolts and corresponding nuts. In one aspect, the spacers 245 can be washers installed on the bolts. In a further aspect, the fasteners and spacers can be formed of UHMW.

When the electrolytic oxidation device 200 is placed in a liquid and an electrical charge is created across the anode 230 and cathode 235, ozone and hydroxyl free radicals form in liquid between the anode 230 and the cathode 235.

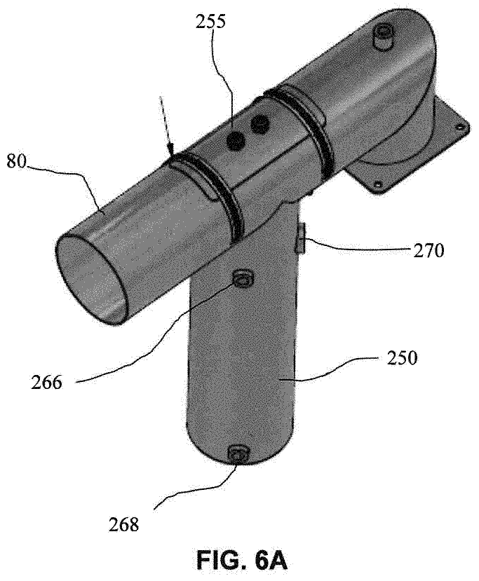

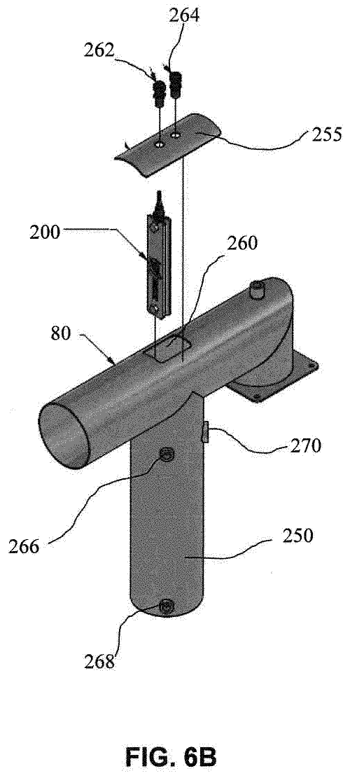

Referring to FIGS. 6A and 6B, the electrolytic oxidation device 200 can be installed in a container 250 positioned beneath the exhaust pipe 80. A cover 255 can be provided on the top of the exhaust pipe 80, covering an opening 260 in the exhaust pipe 80, with the container 250 positioned below and in fluid communication with the exhaust pipe 80. The cover 255 can have a first cable gland 262 for running positive and negative wires through to the electrolytic oxidation device 200 and a second cable gland 264 for holding a conduit to route ozone from the ozone generator 90 into liquid in the container 250.

The container 250 can have a first sight tube connection 266, a second sight tube connection 268 and a water fill point 270. The container 250 can define a reservoir that holds water or some other liquid.

To install the electrolytic oxidation device 200, the cover 255 can be removed to expose the opening 260 and the electrolytic oxidation device 200 lowered into the container 250 through the opening 260. A positive power lead and a negative power lead (not shown) can pass through the first cable gland 262 to supply a voltage to the electrolytic oxidation device 200. The container 250 can then be filled with a liquid, such as up to the water fill point 270, so that the electrolytic oxidation device 200 is suspended in the liquid contained in the container 250.

Ozone can be injected into the liquid through a conduit back to the ozone generator 90 and passing through the second cable gland 264 into the container 250.

When the ozone generator 10 is operating and air is circulating through the system 10 with ozone being added to the air being discharged through the exhaust tube 80 by the ozone generator 90, an electrical charge can be provided to the electrolytic oxidation device 200 suspended in liquid in the container 250, along with ozone being introduced into the liquid. The electrolytic oxidation 200 generates ozone and hydroxyl free radicals in the liquid in the container and this ozone and hydroxyl free radicals can rise to the exhaust pipe 80, which also has ozone added to it by the ozone generator 90. This air can then be directed out the exhaust vent 42 to mix with the air in the enclosed space. This adds more ozone and hydroxyl free radicals to the air in the enclosed space, but vaporized liquid from the container 250 can also increase the humidity in the air in the enclosed space helping with the disinfection/virus deactivation of the enclosed space by the ozone generated by the system 10.

Referring again to FIGS. 1-4, the controller 300 can be used to control the operation of the system 10 and the control panel 150 can be operatively connected to the controller 300 to control the operation of the controller 300. The controller 300 can control the operation of the blower 60, the ozone generator 90, the oxygen generator 82 and the UV light 52. The controller 300 can also be connected to the ozone sensor 84 to obtain ozone measurements for the air in the enclosed space. The control panel 150 can be used to control the controller 300 and therefore the operation of the system 10.

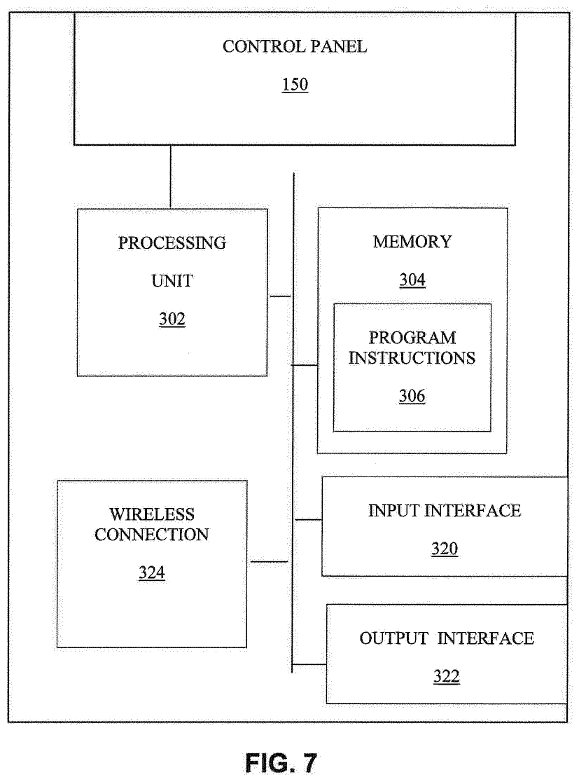

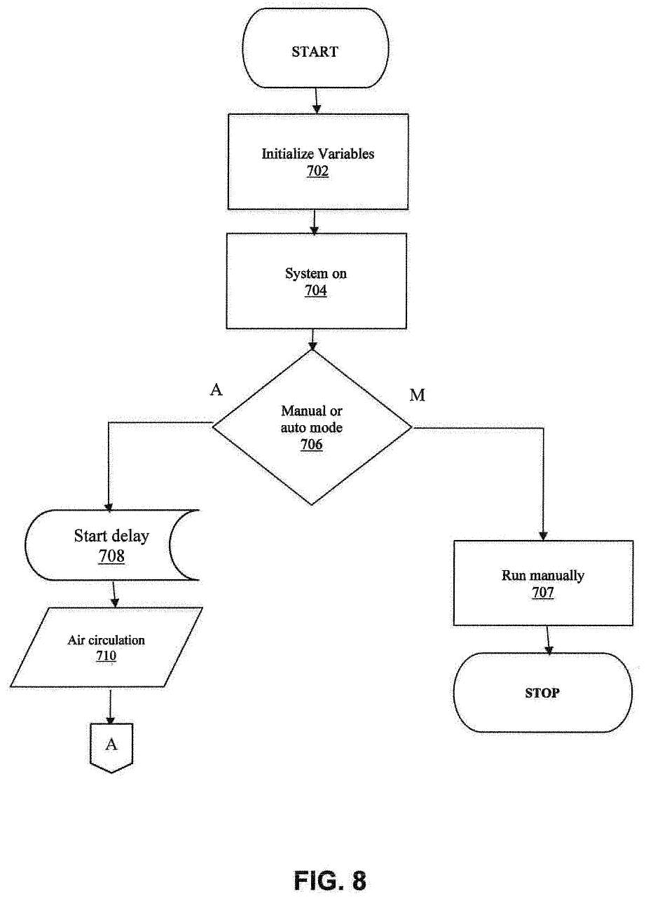

FIG. 7 shows a schematic illustration of the controller 300 in one aspect. The controller 300 can include a processing unit 302, such as a microprocessor that is operatively connected to a computer readable memory 304 and can control the operation of controller 300. Program instructions 306, for controlling the operation of the processing unit 302, can be stored in the memory 304 as well as any additional data needed for the operation of the controller 300.

An input interface 320 can be provided operatively connected to the processing unit 302 so that the controller 300 can receive signals from external sensors. In this manner, the controller 300 can be connected to the ozone sensor 84 to detect the amount of ozone in the air surrounding the system 10.

An output interface 322 can be provided operatively connected to the processing unit 302 to send signals to other devices in the system 10. The output interface 322 can be connected to the UV light 52 to turn the UV light 52 on and off to selectively illuminate the UV light chamber 50. The output interface 322 can also be connected to blower 60 to turn the blower 60 on and off to selectively circulate air through the system 10. The output interface 322 can also be connected to the ozone generator 90 to selectively start and stop the ozone generator 90 from generating ozone.

The control panel 150 can be provided operatively connected to the controller 300 to allow a user to enter inputs into the controller 300 and control it.

In one aspect, the controller 300 can include a wireless connection 324, such as a Bluetooth™ connection or an 802.11 connection, that can allow a device such as smart phone or tablet to be connected wirelessly to the controller 300 and be used to control the controller 300 instead of the control panel 150. In one aspect, the interface device could be a smart phone or tablet running an application (app), allowing an operator of the system 10 to use his or her smart phone or tablet to control the system 10.

To achieve proper deactivation of SARS-COVID-2 or other viruses or contaminants, it is necessary to achieve an ozone concentration in the enclosed space for an effective treatment time period. Alternatively, if the ozone is being used for pest control, the ozone concentration achieved and the treatment time period used should be effective for eliminating the desired pests. Higher concentrations of ozone typically require lower treatment time periods, but lower concentrations of ozone can also be effective with longer treatment time periods. After this treatment time period is finished, the system 10 can use the UV light 52 to remove ozone from the air in the enclosed space until the ozone has been reduced to a safe level allowing people to re-enter the enclosed space.

In operation, the system 10 can be used to treat an enclosed space, such as a room, to disinfect it for viruses, such as SARS-COVID-2. The system 10 can be wheeled into the enclosed space, such as a room, that the operator desires to treat and the room secured so that no persons can enter the room while the system 10 is in use.

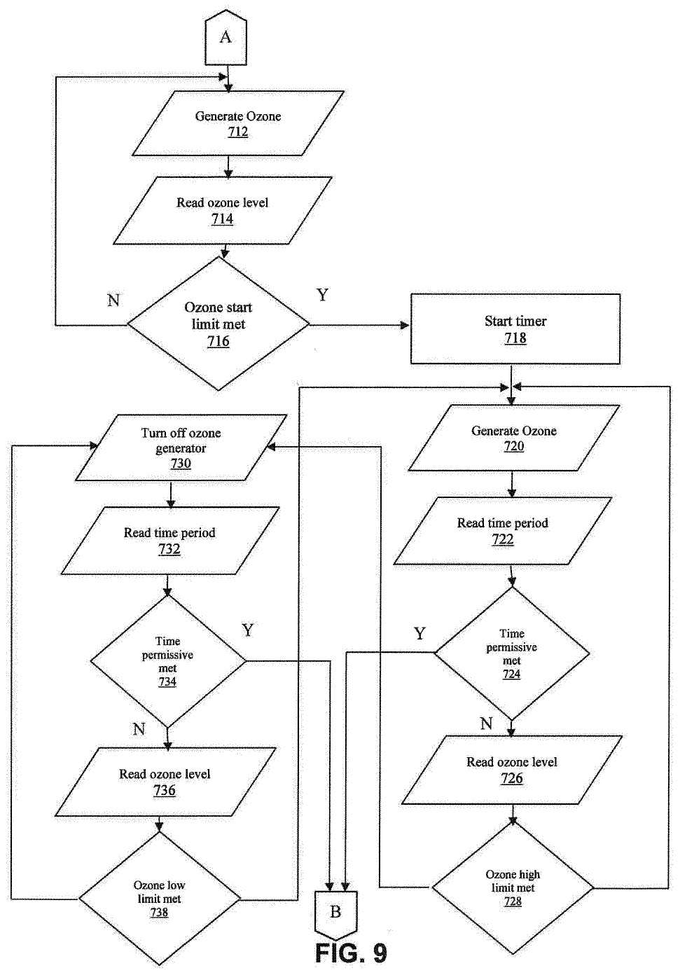

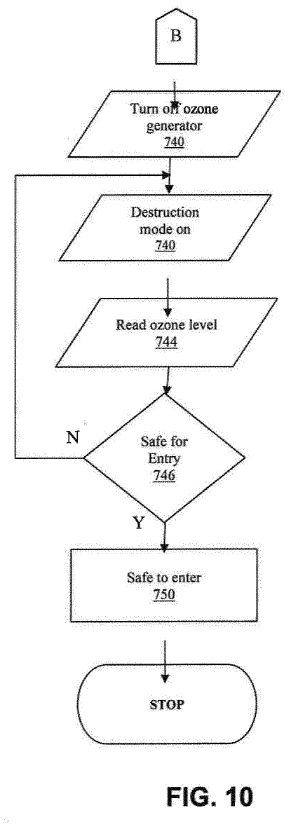

FIGS. 8-10 show a flowchart of a method 700 that can be performed by the controller 300 for using the system 10 to treat SARS-COVID-2, as well as other viruses, in the enclosed space surrounding the system 10. In one aspect, the system 10 can have an automatic mode that follows a defined method and does not require a lot of interaction by the operator after the method is started. The system 10 could also be operated in a manual mode allowing an operator to manually turn on and start the system and bypassing any permissives and interlocks.

When the system 10 is started, the controller 300 can, in response to the program instructions 306 in the computer readable memory 304, start and begin the method with step 702 and the operator initializing the variables to be used by the system 10. These variables can be the set points used by the system 10, such as a start delay time period, a treatment time period, a set ozone target level, an ozone high limit, an ozone low limit, etc. In addition, these variables can be such that a dedicated start and stop time can be used for operation during certain periods of the day outside of set business or office hours.

With the variables initialized at step 702, the controller 300 can move onto step 704 and turn the system 10 on. This can involve having the operator selecting whether they want the system 12 to run in automatic mode or manual mode. In automatic mode, the controller 300 can cause the system 12 to automatically run through a process of disinfecting the enclosed space with ozone with little or no input from a human operation. In manual mode, a human operator can control the operation of the system 12 with the controller 300 merely implementing the instructions received from the human operator.

The controller 300 can then check at step 706, whether the controller 300 is going to operate the system 10 in automatic mode or manual mode. If the controller 300 determines that the operator wants the system 10 to run in automatic mode, the controller 300 can move to step 708 with the system 10 entering the start delay time period. This start delay time period can be a period of time that is long enough for the human operator and anyone else to leave the enclosed space and the enclosed space to be secured to prevent anyone from entering the enclosed space while the system 10 is generating ozone. For example, this start delay time period can be 30 minutes. In another aspect, the operator can set the amount of time for the start delay time period, such as at step 702 of the method.

In one aspect, at step 708, the system 10 can also light the beacon 102 such as a light and have a sounder 104, such as a siren, to indicate to people in the vicinity of the system 10, both visually with the beacon 102 and audibly with the sounder 104, that the system 10 is in operation and starting up to warn them to leave the enclosed space.

With the system 10 started at step 708, the controller 300 can start air circulation through the system 10 at step 710. At step 710, the controller 300 can use the output interface 322 to send a signal to the blower 60 to turn on the blower 60. This will cause air from the enclosed space surrounding the system 10 to be circulated through the system 10 by being sucked into the intake vent 40 and into the UV light box 50. At this point, the UV light 52 provided in the UV light chamber 50 is off so that there is no UV light being shone into the UV light chamber 50 and therefore any ozone in the air passing through the UV light chamber 50 will not be broken down into oxygen by the UV light. The air can be sucked through the UV light chamber 50 and through the intake tube 70 to the blower 60. The blower 60 will then force this air through the exhaust pipe 80 and out the exhaust vent 42 into the enclosed air surrounding the system 10.

With air circulating through the system 10 at step 710, the controller 300 can move onto step 712 and begin generating ozone. The controller 300 can use the output interface 322 to turn on the ozone generator 90 and the oxygen generator 82. This will cause the oxygen generator 82 to start generating oxygen and then routing this oxygen to the ozone generator 90 to start converting this oxygen into ozone. The generated ozone from the ozone generator 90 can then be added to the air circulating through the system 10 by routing it to the inlet port 81 and into the exhaust pipe 80. In one aspect, ozone will not be added to the exhaust pipe 80 until a flow of oxygen from the oxygen generator 82 is detected reaching the ozone generator 90. This will cause the air that is circulating through the system 10 to have ozone that is generated by the ozone generator 90 being added to it, enriching the air circulated through the system 10 with ozone. When the air is discharged from the system 10 through the exhaust vent 42, this ozone enriched air will mix with the air in the enclosed space, increasing the amount of ozone in the air in the enclosed space.

If the system 10 contains an electrolytic oxidation device 200, the controller 300 can also send a signal to the electrolytic oxidation device 200, using the output interface 322, to start the electrolytic oxidation device 200 and start generating ozone and hydroxyl free radicals from the liquid in the container 200 at step 712. Ozone and hydroxyl free radicals generated in the container 250 can enter the exhaust pipe 80 where this ozone and hydroxyls can mix with the air passing through the system 10 to add the hydroxyl free radicals to the air in the enclosed space.

In one aspect, the system 10 can continue to light the beacon 102 and have the sounder 104 to indicate to people in the vicinity of the system 10, that the system 10 is in operation.

The system 10 can use the blower 60 and the ozone generator 90 to enrich the air in the enclosed space with ozone until the ozone in the air in the enclosed space reaches an ozone level sufficient to deactivate airborne viruses and viruses on surfaces like SARS-COVID-2 and other contaminants (i.e. the set ozone target level). The system 10 can then maintain the ozone level in the enclosed space within a desired ozone range for the treatment time period (i.e. between the ozone high limit and the ozone low limit). This treatment time period can be a period of time found to be sufficient to destroy SARS-COVID-2 and other viruses in the desired ozone range. In one aspect, this treatment time period can be 30 minutes. Alternatively, the treatment time period can be entered by the operator at step 702.

After the controller 300 starts having ozone generated at step 712, the controller 300 can move to step 714 and as the blower 60 continues to run and the ozone generator 90 can continue to generate ozone and inject it into the air circulating through the system 10, the controller 300 can obtain a measurement from the ozone sensor 84 of the level of ozone in the enclosed space surrounding the system 10. At step 716, the controller 300 can use the ozone measurement taken from the ozone sensor 84 at step 714 to determine if the ozone in the air surrounding the system 10 has reached the set ozone target level. If at step 716, the ozone measured in the enclosed space has not yet reached the set ozone target level, the controller 300 can return to step 712, and continue to circulate air from the enclosed space through the system 10, adding ozone to the air at step 712, measuring the ozone level in the air in the enclosed space at step 714 and checking at step 716 to see if the ozone has reached the set ozone target level.

Once the set ozone target level has been reached at step 716, the controller 300 can start the timer to begin the treatment time period at step 718 and the move to step 720 where it continues to generate ozone.

If the system 10 contains an electrolytic oxidation device 200, the controller 300 can continue to have the electrolytic oxidation device 200 generating ozone and hydroxyl free radicals from the liquid in the container 250 at step 720 and adding them to the air stream passing through the exhaust tube 80.

After step 722, the controller 300 can move on to step 724 and read the time period. If at step 724, the read time period has not reached the treatment time period, the controller 300 can move to step 726 and obtain a measurement of ozone in the air in the enclosed space using the ozone sensor 84. However, if at step 728, a measured amount of ozone in the air in the enclosed space has not reached the ozone high limit, the controller 300 can return back to step 720 and continue generating ozone, reading the time period at step 722, checking the time reading at step 724 to see if the treatment time period has passed, and if the treatment time period has not passed, obtaining a measurement of the ozone reading in the enclosed space at step 726 and seeing if it has reached the ozone high limit at step 728. The controller 300 can keep following these steps until the ozone in the enclosed space has reached the ozone high limit or the treatment time period has passed.

In one aspect, the ozone high limit can be 4.5 parts per million (ppm) indicating the air in the enclosed space surrounding the system 10 has reached an ozone concentration of 4.5 ppm. In another aspect, the ozone high limit can be 2.6 parts per million (ppm) indicating the air in the enclosed space surrounding the system 10 has reached an ozone concentration of 2.6 ppm, but a longer treatment time may be needed with this lower limit, such as 90 minutes. In further aspect, the ozone high limit can be between 0.5 and 22 ppm with a corresponding treatment time of between 30 minutes and 360 minutes, with a longer treatment time being used for a lower ozone high limit and a shorter treatment time for a higher ozone high limit.

When the ozone in the enclosed space has reached the ozone high limit, the controller 300 can turn off the ozone generator 90 to stop generating more ozone to keep the ozone in the enclosed room in the desired ozone range. However, some of this ozone in the enclosed space will degrade naturally, from sunlight, on other organics present in the unoccupied space, etc. so the controller 300 can keep monitoring the ozone level in the enclosed space to keep it in the desired ozone range. From step 728, with the controller 300 determining that the ozone in the enclosed space has reached the ozone high limit, the controller 300 can move to step 730 and send a signal through the output interface 322 to the ozone generator 90 to turn off the ozone generator 90, which will stop generating ozone and adding it to the air circulating through the system 12.

If the system 10 contains an electrolytic oxidation device 200, the controller 300 can turn off the electrolytic oxidation device 200 at step 730.

With the ozone generator 90 stopped at step 730, the controller 300 can move onto step 732 and read the time period before checking at step 734 to see if the treatment time period has passed. If the treatment time period has not passed at step 734, indicating more treatment of the enclosed space is desired, the controller 300 can move onto step 736 and use the input interface 320 to obtain a measurement of the ozone in the air in the enclosed space from the ozone sensor 84 before moving to step 738 and determining if the measured ozone in the enclosed space has decreased to the ozone low limit. If at step 738, the measured ozone in the enclosed space has not decreased to the ozone low limit, the controller 300 can keep repeating steps 730, 732, 734, 736 and 738, keeping the system 12 from generating ozone and adding it to the air in the enclosed space, until the ozone has decreased to the ozone low limit (or the treatment time period has ended).

With the ozone generator 90 turned off and the air circulating through the system 10 no longer being enriched with ozone, the ozone in the air of the enclosed space surrounding the system 10 will gradually decrease as the ozone in the air degrades from UV light and continued oxidation on surfaces and airborne aerosols in the enclosed space, etc. When the controller 300 obtains a measurement from the ozone sensor 84 at step 738, that indicates the ozone measurement has reached the ozone low limit, the controller 300 can have the system 12 start generating ozone again and adding it to the air in the enclosed space. The controller 300 can move to step 720 and turn on the ozone generator 90 before once again moving to steps 722, 724, 726 and 728. These steps can then be repeated until the measured ozone in the enclosed spaces reaches the high level again at step 728 (or the treatment time period has been reached at step 724).

In one aspect, the ozone low level can be 4.0 ppm million indicating the air in the enclosed space surrounding the system 10 has reached an ozone concentration of 4.0 ppm. With a high limit of 4.5 ppm and a low limit of 4.0 ppm, the system 10 can keep the ozone concentration in the air in the enclosed space substantially between 4.5 ppm and 4.0 ppm and therefore at an effective level to achieve destruction of viruses in the enclosed space. In another aspect, the ozone low level could be 2.4 ppm. However, these levels will be based on the treatment time period and longer treatment time periods could use even lower levels of ozone.

The system 10 can generate ozone using the ozone generator 90 and adding the generated ozone to the air being circulated through the system 10, which is then added to the air in the enclosed space, increasing the amount of ozone air in the enclosed space. As long as the ozone being measured in the enclosed space is below the ozone high limit, the controller 300 can keep air circulating through the system 10 by running the blower 60 and enriching the air with ozone from the ozone generator 90. However, once the controller 300 gets a measurement from the ozone sensor 84 that indicates the ozone in the air surrounding the system 10 has reached the ozone high limit, the controller 300 can turn off the ozone generator 90 while continuing to have the blower 60 on to continue circulating air through the system 10, but not having it enriched with ozone. This ozone generator 90 can remain off until the measured ozone in the enclosed space reaches the ozone low limit, at which point, the ozone generator 90 can once again be turned on to start increasing the levels of ozone in the enclosed space again. The controller 300 can keep turning off and on the ozone generator 90 as necessary to keep the ozone level in the air in the enclosed space between the ozone high limit and the ozone low limit, while the treatment time period is still running. This will keep the ozone concentration in the air in the enclosed space in the desired ozone range substantially between the ozone high limit and the ozone low limit for the treatment time period and allow the ozone enriched air now in the enclosed space enough time to interact with any viruses or other contaminants in the enclosed space and destroy them.

Once the controller 300 determines that the treatment time period has passed at either step 724 or step 734, the system 10 can stop generating ozone and enter an ozone destruction mode to proactively remove ozone from the enclosed space until the concentration of ozone in the air in the enclosed space has once again decreased to a safe level. When the treatment time period has passed, the controller 300 can move to step 740 use the output interface 322 to turn off the ozone generator 90 (if the ozone generator 90 is currently on) and the oxygen generator 82 to stop ozone from being added to the air circulating through the system 10 before moving to step 742 and entering an ozone destruction mode to proactively remove ozone from the air in the enclosed space.

In the ozone destruction mode, the controller 300 can use the output interface 322 to turn on the UV light 52 in the UV light chamber 50 to degrade the ozone in the air passing through the UV light chamber 50 as it circulates through the system 10. During step 742, the blower 60 can remain on to continue circulating air from the enclosed space through the system 10. However, as the ozone enriched air in the enclosed space is drawn back into the system 10 through the intake vent 40, it enters the UV light chamber 50 which is now being subjected to UV light rays from the UV light 52. As the ozone in the ozone enriched air comes into contact with the UV light rays in the UV light chamber 50, the UV light rays cause the ozone in the air to disassociate and break down into oxygen, removing the ozone from the air. This will cause the air being discharged from the system 10 to have the level of ozone decreased or even removed completely.

The air in the enclosed space can continue to be circulated through the system 10 with the UV light 52 turned on and shining into the UV light chamber 50 causing ozone in the air passing through the light chamber 50 to disassociate into oxygen and thereby reducing or even eliminating the ozone in the air. The controller 300 can use the input interface 320 to obtain a measurement of the ozone in the air in the enclosed space from the ozone sensor 84 at step 744 and at step 746 check to see if the ozone in the air has been reduced to a safe level. In one aspect, this safe level could be a measure of 0.1 ppm, but it could vary depending on the jurisdiction and other factors.

If at step 746, the measurements from the ozone sensor 84 indicate the ozone in the air in the enclosed space has not reached a safe level, the controller 300 can keep obtaining measurements of the ozone in the air in the enclosed space from the ozone sensor 84 at step 744 and check these measurements at step 746 to determine if the ozone in the air has reduced to a sufficiently low level that is safe for people to once again enter the enclosed space or even removed entirely.

When at step 746 it is determined that the ozone in the air in the enclosed space has decreased to a sufficiently low level, the controller 300 can deem it safe to enter the enclosed space and at step 750 stop the blower 60 and the UV light 52. At this point, the system 10 has finished its treatment and it is safe for people to once again enter the enclosed space where the system 10 is. If a beacon 102 and sounder 104 have been activated by the controller 300 to warn people that the system 10 was working, the beacon 102 and sounder 104 can be stopped.

The system 10 could also be run in a manual method, if at step 706 the operator has selected manual mode, the controller 300 can move onto step 707 and be run in manual mode, with the operator controlling the operation of the system 10.

The foregoing is considered as illustrative only of the principles of the invention. Further, since numerous changes and modifications will readily occur to those skilled in the art, it is not desired to limit the invention to the exact construction and operation shown and described, and accordingly, all such suitable changes or modifications in structure or operation which may be resorted to are intended to fall within the scope of the claimed invention.

What is claimed is:

1. An ozone generating system for generating ozone in an enclosed space, the system comprising:
a housing having: a front end; a back end; a first side; a second side; a top; a bottom;
an intake vent passing through the housing;
an exhaust vent passing through the housing;
a blower provided in the housing;
an intake pipe provided in the housing and routing air entering the housing through the intake vent to the blower;
an exhaust pipe provided in the housing and operably connected between the blower and the exhaust vent;
an ozone generator provided in the housing and operably connected to the exhaust pipe to route ozone generated by the ozone generator into the exhaust pipe; and
an ozone sensor operably connected to the controller and operative to measure an amount of ozone in the air surrounding the ozone sensor.

2. The system of claim 1 wherein the housing comprises a removable first access panel on the first side and a removable second access panel on the second side.

3. The system of claim 1 further comprising: swivel casters attached to the bottom of the housing; and, a handle attached to the housing.

4. The system of claim 1 further comprising an inlet port connected to the exhaust pipe, the ozone generator operably connected to the exhaust pipe by the inlet port.

5. The system of claim 1 further comprising a beacon having a visual warning to indicate when the system is operating.

6. The system of claim 1 further comprising a sounder having an audible warning to indicate when the system is operating.

7. The system of claim 1 further comprising an electrolytic oxidation device operative to generate ozone and hydroxyl free radicals and direct the generated ozone and free hydroxyl radicals into the exhaust pipe.

8. The system of claim 7 wherein the ozone and free hydroxyl radicals generated by the electrolytic oxidation device pass into the exhaust pipe with vaporized liquid.

9. The system of claim 7 wherein the electrolytic oxidation device comprises: a positive terminal; a negative terminal; a front cover; a back cover; an anode connected to the positive terminal; a cathode connected to the negative terminal; fasteners securing the front cover, anode, cathode and back cover in series; and spacers provided between the anode and cathode to space apart the anode and the cathode.

10. The system of claim 9 wherein the front cover and the back cover of the electrolytic oxidation device are formed of ultra-high-molecular-weight polyethylene.

11. The system of claim 9 wherein spacers are provided between the front cover and the anode and the cathode and the back cover.

12. The system of claim 9 further comprising a container defining a reservoir that holds liquid and the electrolytic oxidation device, the container positioned below and in fluid communication with the exhaust pipe.

13. The system of claim 1 wherein the intake vent and the exhaust vent are positioned in the front end of the housing.

14. The system of claim 1 further comprising an oxygen generator provided in the housing to generate oxygen and supply the oxygen to the ozone generator.

15. The system of claim 1 further comprising: a UV light chamber provided in the housing between the intake vent and the blower so that air entering the housing through the intake vent passes through the UV light chamber before being directed by the intake pipe to the blower, the UV light chamber having a UV light positioned to illuminate an inside of the UV light chamber.

16. The system of claim 1 further comprising a controller having:
at least one processing unit;
an input interface operatively connected to the ozone sensor;
an output interface operatively connected to: the blower; and the ozone generator; and
at least one memory containing program instructions.

17. The system of claim 16 further comprising a control panel operatively connected to the controller.

18. The system of claim 16 wherein the at least one processing unit is, responsive to the program instructions, operative to:
initiate a start delay time;
send a signal to the blower to turn on the blower and circulate air from the enclosed space surrounding the system through the system before exhausting the air to the enclosed space;
when the start delay time has passed, send a signal to the ozone generator to turn on the ozone generator and routing ozone to the air circulating through the system to form ozone enriched air;
continuing to enrich air circulating through the system with ozone and exhausting the ozone enriched air to the enclosed space, until a signal is received from the ozone sensor indicating an ozone level in the air in the enclosed space has reached a set ozone target level;
maintaining an ozone level in the air in the enclosed space in a desired ozone range for a treatment time period; and
after the treatment time period has passed, sending a signal to the ozone generator to turn off the ozone generator.

19. The system of claim 18 wherein the output interface is connected to a beacon having a visual warning and wherein the at least one processing unit sends a signal to the beacon during operation of the system to provide the visual warning when the system is operating.

20. The system of claim 18 wherein the output interface is connected to a sounder having an audible warning and wherein the at least one processing unit sends a signal to the sounder during operation of the system to provide the audible warning when the system is operating.

21. The system of claim 18 wherein the output interface is connected to an electrolytic oxidation device operative to generate ozone and hydroxyl free radicals and wherein the at least one processing unit sends a signal to the electrolytic oxidation device when the ozone generator is on to generate ozone and hydroxyl free radicals and add the generated ozone and hydroxyl free radicals to the air circulating through the system.

22. The system of claim 18 wherein the ozone level in the air in the enclosed space is maintained in the desired ozone range for the treatment time period by:
continuing to enrich air circulating through the system with ozone and exhausting the ozone enriched air to the enclosed space until a signal is received from the ozone sensor indicating an ozone level in the air in the enclosed space has reached an ozone high limit;
after the ozone level in the air in the enclosed space has reached an ozone high limit, stopping the ozone generator to stop enriching the air circulating through the system with ozone; and
when a signal is received from the ozone sensor indicating an ozone level in the air in the enclosed space has reached an ozone low limit, sending a signal to the ozone generator to turn on the ozone generator and routing ozone to the air circulating through the system to form ozone enriched air.

23. The system of claim 22 wherein the ozone high limit is 4.5 ppm and the ozone low limit is 4.0 ppm.

24. The system of claim 22 wherein the ozone high limit is 2.6 ppm and the ozone low limit is 2.4 ppm.

25. The system of claim 18 wherein the at least one processing unit is further operative to:
after the treatment time period has ended, entering an ozone destruction mode and removing ozone from the air circulating through the system until the ozone in the air in the enclosed space has decreased to a safe level.

26. The system of claim 25 further comprises: a UV light chamber provided in the housing between the intake vent and the blower so that air entering the housing through the intake vent passes through the UV light chamber before being directed by the intake pipe to the blower, the UV light chamber having a UV light positioned to illuminate an inside of the UV light chamber, wherein the output interface of the controller is connected to the UV light, and wherein the ozone destruction mode comprises turning on the UV light in the UV light chamber.

27. The system of claim 26 wherein the safe level is 0.1 ppm or less.

28. A method of generating ozone in an enclosed space, the method comprising
after a start delay time period, adding ozone to air in an enclosed space to form ozone enriched air;
continuing to enrich the air in an enclosed space with ozone, until the ozone in the air in the enclosed space reaches a set ozone target level;
after the ozone in the air in the enclosed space reaches the set ozone target level, maintaining an ozone level in the air in the enclosed space in a desired ozone range for a treatment time period; and
after the treatment time period has passed, stopping adding any ozone to the air in the enclosed space.

29. The method of claim 28 further comprising generating hydroxyl free radicals and adding hydroxyl free radicals and vaporized liquid to the air in the enclosed space.

30. The method of claim 28 wherein the ozone level in the air in the enclosed space is maintained in the desired ozone range for the treatment time period by:
continuing to enrich the air in the enclosed space until an ozone level in the air in the enclosed space has reached an ozone high limit;
after the ozone level in the air in the enclosed space has reached an ozone high limit, stop enriching the air in the enclosed space with ozone; and
when an ozone level in the air in the enclosed space has reached an ozone low limit, enriching the air in the enclosed space with ozone.

31. The method of claim 30 wherein the ozone high limit is 4.5 ppm and the ozone low limit is 4.0 ppm.

32. The method of claim 30 wherein the ozone high limit is 2.6 ppm and the ozone low limit is 2.4 ppm.

33. The method of claim 3 wherein after the treatment time period has ended, removing ozone from the air in the enclosed space until the ozone in the air in the enclosed space has decreased to a safe level.

34. The method of claim 33 wherein the safe level is 0.1 ppm or less.
